(19) United States
(12) Patent Application Publication
(10) Pub. No.: US 2025/0073484 A1
(43) Pub. Date: Mar. 6, 2025

(54) MEDICAL DEVICE WITH CONTACTLESS DATA TRANSMISSION

(71) Applicant: Siemens Healthineers AG, Forchheim (DE)

(72) Inventor: Marcel RUF, Langensendelbach (DE)

(73) Assignee: Siemens Healthineers AG, Forchheim (DE)

(21) Appl. No.: 18/816,139

(22) Filed: Aug. 27, 2024

(30) Foreign Application Priority Data
Aug. 30, 2023 (DE) 10 2023 208 317.5

(51) Int. Cl. A61N 5/06

(52) U.S. Cl. CPC A61N 5/06; A61N 2005/0626; A61N 2005/0632; A61N 2005/0658

(57) ABSTRACT

A medical device has a base body on a delimiting surface of a room. The medical device furthermore has an additional body supported pivotably about a pivot axis on the base body. Thus, by pivoting the additional body about the pivot axis, it is possible to set a rotational position of the additional body relative to the base body. The additional body directly or indirectly carries a radiation source for emitting ionizing radiation. A data transmission element is on each of the base body and on the additional body in each case. The data transmission elements are arranged flush on the base body and on the additional body with respect to the pivot axis and are spaced apart from one another, seen in the direction of the pivot axis.

### CROSS-REFERENCE TO RELATED APPLICATION(S)

The present application claims priority under 35 U.S.C. § 119 to German Patent Application No. 10 2023 208 317.5, filed Aug. 30, 2023, the entire contents of which is incorporated herein by reference.

### FIELD

One or more example embodiments relates to a medical device with contactless data transmission.

### RELATED ART

Computed tomography units can be constructed such as disclosed in DE 10 2021 213 027 A1. With a computed tomography unit, the pivot movement differs to the extent that, with a computed tomography unit, the additional part (the gantry) often executes a number of complete rotations about the “pivot axis”, i.e. is not so much pivoted as rotated instead.

Computed tomography units are not the only medical devices in which an additional body is supported for rotational movement on a base body. Examples of such further medical devices are a C-arm system, a linear accelerator for radiotherapy and a mammography device. The present invention relates to this type of medical devices.

With such medical devices too it is necessary to exchange data between the (more or less stationary) base body and the (pivotable) additional body. Usually this data transmission takes place via data lines.

It is also known to use a standardized wireless transmission, for example WIFI or Bluetooth, instead of a line-based data transmission.

### SUMMARY

If a data transmission takes place via data lines then the pivot angle by which the additional body can be pivoted relative to the base body is restricted. Furthermore wear occurs during each pivot movement-even if said wear is only very small during the respective pivot movement. Although standardized wireless transmission avoids the disadvantages of the restricted pivot angle and of the wear that occurs, it is significantly more expensive and, what is more, must fulfill the legal requirements of wireless connections. Therefore such methods are not used or are used only rarely in practice.

One or more example embodiments creates opportunities via which the disadvantages of the prior art can be avoided.

The is achieved by a medical device with the features of claim 1. Advantageous embodiments of the inventive medical device are the subject matter of the dependent claims 2 to 8.

### BRIEF DESCRIPTION OF THE DRAWINGS

The characteristics, features and advantages, as well as the manner in which these are achieved, will become clearer and easier to understand in conjunction with the description of the exemplary embodiments given below, which will be explained in greater detail in conjunction with the drawings. In the figures, in schematic diagrams:

FIG. 1 shows a medical device according to one or more example embodiments,

FIG. 2 shows a detail from FIG. 1 in the transition area between a base body and an additional body of the medical devices in cross-section according to one or more example embodiments,

FIG. 3 shows a view from above of a data transmission element of the base body, seen from the additional body according to one or more example embodiments,

FIG. 4 shows a view from above of a data transmission element of the additional body, seen from the base body, according to one or more example embodiments,

FIG. 5 shows a radiation lobe according to one or more example embodiments,

FIG. 6 shows a block diagram of data transmission elements according to one or more example embodiments, and

FIG. 7 shows a further block diagram of data transmission elements according to one or more example embodiments.

### DETAILED DESCRIPTION

In accordance with one or more example embodiments, a medical device of the type stated at the outset is embodied such that the data transmission elements on the base body and on the additional body are arranged flush with regard to the pivot axis and, seen in the direction of the pivot axis, are spaced apart from one another.

One or more example embodiments, as already mentioned, involves medical devices such as for example a C-arm system, a linear accelerator for radiotherapy and a mammography device.

The delimiting surfaces of the room are the walls, the ceiling and the floor of the room. The arrangement on the corresponding delimiting surface is produced entirely naturally, because the medical device cannot float in the room.

The base body can be arranged in a fixed location on the corresponding delimiting surface. This is in particular often the case for an arrangement on a wall of the room. But this is also possible for an arrangement on the celling or on the floor of the room. As an alternative the base body can be arranged movably on the corresponding delimiting surface. For example the base body can have castors, via which the base body stands on the floor of the room, so that the medical device is able to be moved on the floor of the room using them. The base body can also be able to be moved along a guide element similar to rails, which for its part is connected to the corresponding delimiting surface. In the case of the attachment of the rail-like guide element to a wall of the room, a height adjustment can in particular be possible in this case, in the case of attachment to the ceiling of the room, a positioning in the direction of the pivot axis.

Especially in the case of a C-arm system and a mammography device, the medical device can alternatively be arranged in a fixed location on the corresponding delimiting surface or can be mobile. A linear accelerator is often arranged in a fixed location.

The radiation source for emitting ionizing radiation is often an x-ray source. It can however also involve a linear accelerator or a radiation source that contains a radioactive material.

The transmitted data can for example be control data for the radiation source. In this case the data is transmitted from the base body to the additional body. As an alternative or in addition the transmitted data can for example comprise data acquired for measurement of operating data of the radiation source or image data acquired via an x-ray detector arranged on the additional body. In this case the data is transmitted from the additional body to the base body.

The type of carrier signal used for the data transmission can be as required. In an individual case a use of an ultrasound signal is also conceivable. As a rule however the data transmission elements are embodied in such a way that they transmit the data by modulation of an electromagnetic carrier signal.

In some cases it can be necessary to provide a metal screening around the data transmission elements. This reliably avoids any problems that could otherwise occur due to an emission of the carrier signal to the outside. In many cases however such screening is not necessary.

In the case of an electromagnetic carrier signal it is possible for the electromagnetic carrier signal to be a light signal. The light signal can be visible light (wavelength approx. 400 nm to approx. 750 nm), infrared light (wavelength 750 nm to 1 mm) or in individual cases also ultraviolet light (wavelength approx. 400 nm to approx. 100 nm).

One alternative, which is currently preferred, is for the electromagnetic carrier signal to be an electromagnetic signal in the narrower sense, i.e. that the electromagnetic carrier signal has a wavelength of at least 3 mm, in particular of at least approx. 5 mm. A wavelength of 3 mm corresponds to a frequency of the carrier signal of approx. 100 GHz, a wavelength of approx. 5 mm to a frequency of the carrier signal of approx. 60 GHz.

On the other hand the electromagnetic carrier signal preferably has a wavelength of maximum 20 mm, in particular of maximum 10 mm. A wavelength of 20 mm corresponds to a frequency of the carrier signal of approx. 15 GHZ, a wavelength of approx. 10 mm to a frequency of the carrier signal of approx. 30 GHz.

It is quite especially preferred for the electromagnetic carrier signal to have a wavelength at which the carrier signal interacts with oxygen or nitrogen. At such wavelengths or frequencies oxygen and nitrogen have a clear attenuating action. The carrier signal is thereby rapidly attenuated in the air, so that the range in air is limited due to physical circumstances. With use of such a carrier frequency it goes without saying that the transmitted signal is very immune to interference and very immune to listening-in. In particular such a frequency (a so-called absorption line for oxygen) exists at around 60 GHz.

Preferably the electromagnetic carrier signal is circularly polarized. The particular effect of the circular polarization of the electromagnetic carrier signal is that the data transmission between the two data transmission elements is independent of the extent to which the additional body is pivoted relative to the base body about the pivot axis.

In the case of a light signal, a circular polarization can be achieved for example by the light signal first passing through a polarization filter within the data transmission element acting as the transmitter, so that after the polarization filter it is first linearly polarized. In this case a quarter wave plate can be arranged downstream of the polarization filter, which converts the linearly polarized light into circularly polarized light. For an electromagnetic signal, corresponding data transmission elements, in which the electromagnetic carrier signal has a wavelength of between 3 mm and 20 mm and the radiation is circularly polarized, are commercially available. For example a corresponding transmit and receive arrangement made by Rosenberger Hochfrequenztechnik GmbH & Co. KG, 83413 Fridolfing, Germany, is sold under the product name RoProxCon.

Usually a number of electrical energy consumers are arranged on the additional body. The energy consumers can in particular comprise the radiation source. Other energy consumers-be they in addition or be they alternative-are also possible however. The energy consumers are connected via lines arranged in the additional body to additional body-side energy transmission elements. In a similar way an energy supply of the room is connected via lines arranged in the base body to base body-side energy transmission elements. The energy transmission elements for their part are coupled to one another for transmission of energy from the base body to the additional body. Preferably the energy coupling of the energy transmission elements to one another is independent of the rotational position of the additional body relative to the base body. The formulation “independent of the rotational position of the additional body relative to the base body” is not intended to mean that the additional body is only able to be pivoted relative to the base body over a specific and thereby restricted range of angles about the pivot axis and the coupling for energy is provided in each rotational position within the range of angles. Instead, the formulation means that the additional body can be brought into any given rotational position relative to the base body (i.e. over the full 360°) and the coupling for energy is then also provided in each rotational position within the range of angles. The coupling for energy thus does not represent any limitation for the pivoting of the additional body relative to the base body, so that the additional body could in principle be pivoted ever further, even in principle through any given number of complete rotations about the pivot axis.

This means that a complete freedom for pivoting of the additional body relative to the base body exists. The pivot movement is thus—unlike with a connection via cable—no longer restricted. Advantages in the workflow are produced thereby.

The interface is embodied in the simplest case as a number of slip rings. A contactless transmission via a magnetic coupling similar to a transformer is also possible. The primary winding and the secondary winding of the “transformer” can be embodied as a type or ring in this case and be arranged concentrically to one another, so that the two windings, seen in the direction of the pivot axis, are arranged at the same height and seen orthogonally to the pivot axis, the one winding surrounds the other winding radially to the outside. As an alternative the two windings can for example each be embodied in a ring shape circulating at the same distance about the pivot axis and seen in the direction of the pivot axis, be at a small distance from one another. In both cases the magnetic coupling can be strengthened by an iron core.

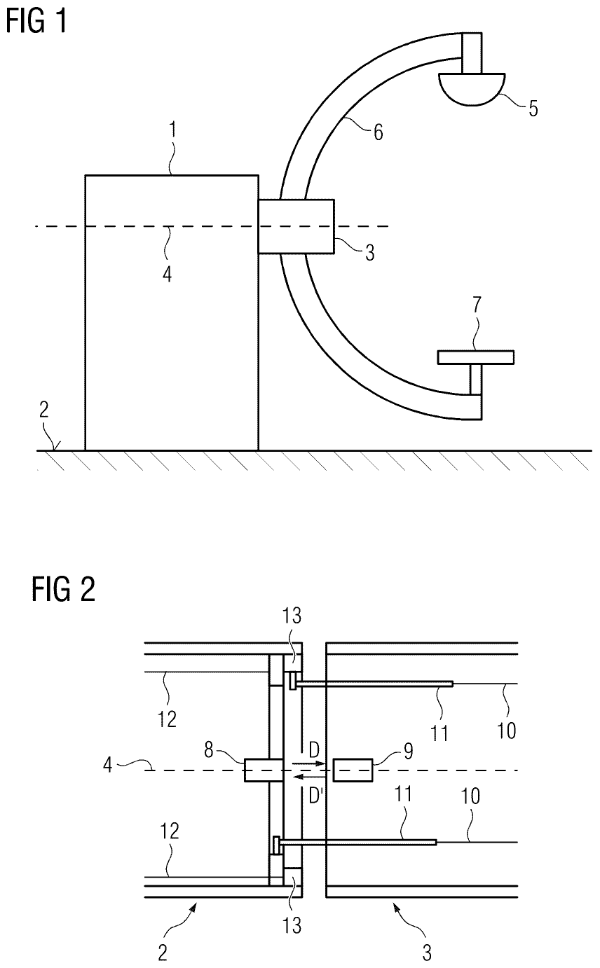

In accordance with FIG. 1 a medical device has a base body 1. The base body 1 is arranged on a delimiting surface 2 of a room. In the present example the delimiting surface 2 is the floor of the room. As an alternative it could also involve a wall or a celling of the room however. In the present example the base body 1 is furthermore arranged in a fixed position (stationary) on the delimiting surface 2. It could however also be arranged movably (mobile, drivable) of the delimiting surface 2.

The medical device furthermore has an additional body 3. The additional body 3 is pivotably supported on the base body 1. The additional body 3 is thus able to be pivoted about a pivot axis 4 relative to the base body 1. The pivoting of the additional body 3 about the pivot axis 4 thus enables a rotational position of the additional body 3 to be set relative to the base body 1.

Where the terms axial, radial and tangential are used below, they are always related to the pivot axis 4. Axial is a direction parallel to the pivot axis 4. Radial is a direction orthogonal to the pivot axis 4 directly towards the pivot axis 4 or away from it. Tangential is a direction that, with a constant axial position and with a constant radial distance, is directed in the shape of a circle about the pivot axis 4.

The additional body 3 directly or indirectly carries a radiation source 5 for emitting ionizing radiation. In the present example the radiation source 5 is an x-ray source that is an element of a C-arm 6. Thus the additional body 3 carries the radiation source 5 indirectly. The C-arm 6 furthermore carries an x-ray detector 7. The medical device is therefore embodied in the present example as a C-arm system. Other embodiments of the medical device are also possible however, for example as a mammography device or as a radiotherapy device.

Arranged on the base body 1 and on the additional body 3 in each case in accordance with FIG. 2 is a transmission element 8, 9. Via the transmission elements 8, 9, data D, D′ is able to be transmitted in a contactless manner from the base body 1 to the additional body 3 and/or from the additional body 3 to the base body 1. In the present example the data D is transmitted from the base body 1 to the additional body 3, the data D′ from the additional body 3 to the base body 1. The transmission element 8 thus functions as transmitter for the data D, while the transmission element 9 functions as receiver. The converse is true for the data D′.

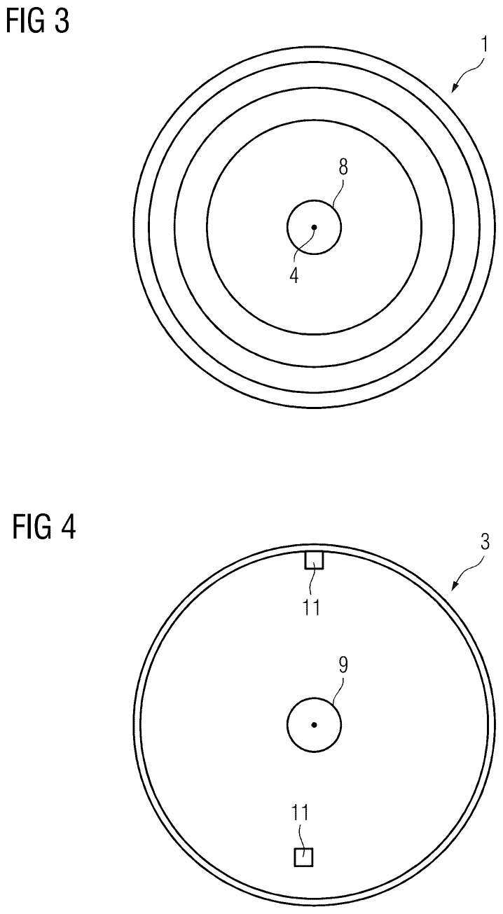

In accordance with FIGS. 2 to 4 the transmission elements 8, 9 on the base body 1 and on the additional body 3 are arranged flush in relation to the pivot axis 4. The transmission elements 8, 9 are thus arranged, seen in a plane orthogonal to the pivot axis 4, directly on the pivot axis 4. Furthermore the transmission elements 8, 9 in accordance with FIG. 2, seen in the direction of the pivot axis 4, are spaced apart from one another. The space between them is quite small as a rule and lies in the range of single millimeters to tens of millimeters.

FIG. 5 shows a preferred emission characteristic with which the transmission element 8, 9 functioning as the transmitter in each case radiates a carrier signal, via which the data D, D′ is transmitted. In accordance with FIG. 5 the respective transmitter essentially transmits in the direction of the pivot axis 4, thus has a narrow radiation lobe. In concrete terms the radiation intensity has fallen, at the latest at an angle of 30° to the pivot axis 4, to ¼ of the value with which the respective transmitter is radiating in the direction of the pivot axis 4. There are however also emission characteristics possible for which the emission lobe has a greater opening angle. In any event however as a rule the transmission element 8, 9 functioning as a receiver in each case has a corresponding sensitivity in relation to the receipt.

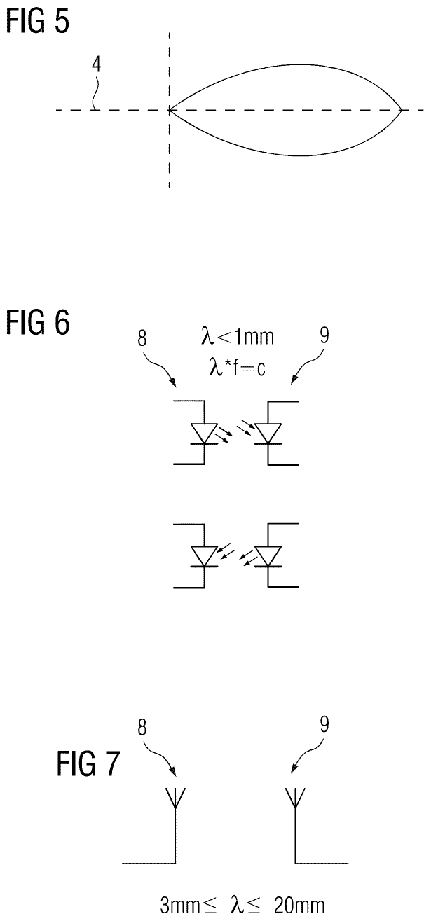

Preferably the transmission elements 8, 9 in accordance with FIGS. 6 and 7 are embodied in such a way that they transmit the data D, D′ by modulation of an electromagnetic carrier signal.

In the embodiment in accordance with FIG. 6 the electromagnetic carrier signal is a light signal. This is indicated in FIG. 6 by a value of less than 1 mm being specified as the wavelength λ of the carrier signal and the light speed c as the product of the wavelength λ and the frequency f of the carrier signal. 1 mm is usually seen as the boundary between infrared on the one side and “normal” electromagnetic waves on the other side.

In the embodiment in accordance with FIG. 7 the carrier signal is a “normal” electromagnetic carrier signal. It thus has a wavelength λ of at least 3 mm. On the other hand the electromagnetic carrier signal is still very high frequency. In concrete terms a wavelength λ of maximum 20 mm, preferably of maximum 10 mm. In particular the electromagnetic carrier signal can have a wavelength λ of approx. 5 mm. This corresponds, for an electromagnetic carrier signal, to a frequency f or approx. 60 GHz. In actual fact the frequency f can lie between 61 GHz and 61.5 GHz. In this case the frequency f lies in an ISM band. It can however also lie at a frequency of approx. 60 GHz, at which oxygen interacts with the carrier signal.

The electromagnetic carrier signal is preferably circularly polarized. Possibilities for generating a circularly polarized electromagnetic carrier signal are known to persons skilled in the art.

A number of electrical energy consumers are arranged on additional body 3. As a rule the radiation source 5 is such a consumer. If present, the x-ray detector 7 is also such a consumer. The energy consumers are connected to the additional element-side energy transmission elements 11 via lines 10 arranged in the additional body 3 (see FIG. 2). In a similar way the energy supply of the room is connected, via lines 12 arranged in the base body 1, to base body-side energy transmission elements 13. The additional body-side energy transmission elements 11 and the base body-side energy transmission elements 13 are coupled to one another for energy transmission from the base body 1 to the additional body 3. The energy coupling of the energy transmission elements 11, 13 to one another is independent of the rotational position of the additional body 3 relative to the base body 1. According to the diagram depicted in FIG. 2 for example, the base body-side energy transmission elements 13 are embodied as slip rings and the additional body-side energy transmission elements 11 are embodied as capture elements that slide on the slip rings.

In summary the present invention thus relates to the following subject matter:

A medical device has a base body 1, which is arranged on a delimiting surface 2 of a room. The medical device furthermore has an additional body 3, which is supported on the base body 1 so that it can be pivoted about a pivot axis 4. Thus, by pivoting of the additional body 3 about the pivot axis 4, it is possible to set a rotational position of the additional body 3 relative to the base body 1. The additional body 3 directly or indirectly carries a radiation source 5 for emitting ionizing radiation. Arranged on the base body 1 and on the additional body 3 in each case is a data transmission element 8, 9. Via the data transmission elements 8, 9 data D, D′ is able to be transmitted in a contactless manner from the base body 1 to the additional body 3 and/or from the additional body 3 to the base body 1, so that, with regard to a respective data transmission, one of the. data transmission elements 8, 9 functions as a transmitter and as a receiver in each case. The data transmission elements 8, 9 are arranged on the base body 1 and on the additional body 3 flush in relation to the pivot axis 4 and spaced apart from one another, seen in the direction of the pivot axis 4.

The present invention has many advantages. The data transmission can be realized at low cost, reliably and robustly. With a suitable design it does not represent a wireless connection in the legal sense, so that no wireless approval is needed. High data rates of several Gigabits pro second can be realized. Unidirectional or bidirectional data transmission is possible, as required. The required adjustment accuracy of the transmission elements 8, 9 is very small. In particular in the case of using an electromagnetic carrier signal with a wavelength λ of between 3 mm and 20 mm, it also does not represent a problem for a partition plate or the like to be arranged between the data transmission elements 8, 9, provided the partition plate is not electrically conductive. This can be of importance for hermetic sealing. A complete freedom of pivot movement is produced. Wear does not occur. Due to the inherently short range of the data transmission there is practically no eavesdropping on the data transmission and practically no interference, so that a high degree of data security is produced.

Although the invention has been illustrated and described in greater detail by the preferred exemplary embodiment, the invention is not restricted by the disclosed examples and other variations can be derived herefrom by the person skilled in the art, without departing from the scope of protection of the invention.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, diagrams, that may be block etc.) implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example memory flash devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A medical device, comprising:
a base body on a delimiting surface of a room;
an additional body supported pivotably about a pivot axis on the base body such that by pivoting of the additional body about the pivot axis, a rotational position of the additional body is set relative to the base body, the additional body directly or indirectly carries a radiation source configured to emit ionizing radiation; and
a data transmission element on each of the base body and the additional body such that data is transmittable in a contactless manner at least one of from the base body to the additional body or from the additional body to the base body via the data transmission elements, one of the data transmission elements is operable as a transmitter and a receiver, wherein
the data transmission elements are arranged flush on the base body and on the additional body with respect to the pivot axis and are spaced apart from one another.

2. The medical device of claim 1, wherein the data transmission elements are configured to transmit the data by modulating an electromagnetic carrier signal.

3. The medical device of claim 2, wherein the electromagnetic carrier signal is a light signal.

4. The medical device of claim 2, wherein the electromagnetic carrier signal has a wavelength of at least 3 mm.

5. The medical device of claim 4, wherein the wavelength is a maximum 20 mm.

6. The medical device of claim 4, wherein the electromagnetic carrier signal has the wavelength at which the carrier signal interacts with oxygen or nitrogen.

7. The medical device of claim 2, wherein the electromagnetic carrier signal is circularly polarized.

8. The medical device of claim 1, further comprising:
a number of electrical energy consumers on the additional body, the energy consumers connected via lines to additional element-side energy transmission elements in the additional body, an energy supply of the room is connected via lines arranged in the base body to base body-side energy transmission elements, the base body-side energy transmission elements configured to transmit energy from the base body to the additional body having an energy coupling to the base body and the additional body and the energy coupling is independent of the rotational position of the additional body relative to the base body.

9. The medical device of claim 5, wherein the electromagnetic carrier signal has the wavelength at which the carrier signal interacts with oxygen or nitrogen.

10. The medical device of claim 9, wherein the electromagnetic carrier signal is circularly polarized.

11. The medical device of claim 10, further comprising:
a number of electrical energy consumers on the additional body, the energy consumers connected via lines to additional element-side energy transmission elements in the additional body, an energy supply of the room is connected via lines arranged in the base body to base body-side energy transmission elements, the base body-side energy transmission elements configured to transmit energy from the base body to the additional body having an energy coupling to the base body and the additional body and the energy coupling is independent of the rotational position of the additional body relative to the base body.
